(19) United States
(12) Patent Application Publication
(10) Pub. No.: US 2024/0090397 A1
(43) Pub. Date: Mar. 21, 2024

(54) TOMATO PLANT HAVING IMPROVED INSECT RESISTANCE

(71) Applicant: ENZA ZADEN BEHEER B.V., Enkhuizen (NL)

(72) Inventors: Teresa Maria Montoro Ponsoda, Enkhuizen (NL); Jan-Willem De Kraker, Enkhuizen (NL); IIona Gertruida Maria Broersen, Enkhuizen (NL); IIja Roobeek, Enkhuizen (NL); Marieke Ykema, Enkhuizen (NL); Fausto Rodriguez Sánchez, Enkhuizen (NL)

(21) Appl. No.: 18/283,629

(22) PCT Filed: Mar. 24, 2021
(86) PCT No.: PCT/EP21/57590

§ 371 Date: Sep. 22, 2023

(51) Int. Cl. A01H 1/00; A01H 6/82

(52) U.S. Cl. CPC A01H 1/127; A01H 6/825

(57) ABSTRACT

The present invention relates to a tomato plant having improved insect resistance, more specifically whitefly or mite resistance, wherein the plant comprises a SlAT2 gene encoding for an acetyl-CoA-dependent acyltransferase enzyme and an AP2e gene encoding for a APETALA2 ethylene-responsive transcription factor. The present invention further relates to methods for providing a tomato plant having improved insect resistance and the use of a SlAT2 gene in combination with an AP2e gene for providing insect resistant tomato plants.

The present invention relates to a tomato plant having improved insect resistance, more specifically whitefly or mite resistance, wherein the plant comprises a SlAT2 gene encoding for an acetyl-CoA-dependent acyltransferase enzyme and an AP2e gene encoding for a APETALA2 ethylene-responsive transcription factor. The present invention further relates to methods for providing a tomato plant having improved insect resistance and the use of a SlAT2 gene in combination with an AP2e gene for providing insect resistant tomato plants.

Whiteflies are of the family Aleyrodidae that typically feed on the abaxial side of plant leaves. There are more than 1500 species been described and especially in warm or tropical climates and in greenhouses, whiteflies present a major problem in crop protection in warm climates and crops grown under greenhouse conditions, resulting in huge economic losses annually worldwide. Many whitefly species are very small in size which complicates their control in greenhouses, and when leaving unchecked whitefly populations in greenhouses rapidly become overwhelming. Whitefly related damage reduces crop quality and quantity, such as a reduction of plant vigour and yield, early wilting, leaf chlorosis and defoliation. The silverleaf whitefly (Bemisia tabaci) is one species of whitefly that are currently one of the most important agricultural pests.

Although several species of whitefly may cause some crop losses simply by sucking sap when they are very numerous, the major harm they do is indirect. The major importance as crop pests is their role as vectors and in the transmission of diseases of plants, including more than 200 plant viruses. Furthermore, whiteflies feed by tapping into the phloem of plants, introducing toxic saliva and decreasing the plants' overall turgor pressure. The whitefly secrete large amounts of honeydew that support harmful infestations of fungi like sooty mold. Since whiteflies congregate in large numbers, susceptible plants can be quickly overwhelmed.

Insecticides such as neonicotinoid, organochlorines, and organophosphate compounds are widely used and are an effective way to control whiteflies. However, continuous application of insecticides results in the development of whiteflies becoming resistant. In addition more environmental friendly, biological methods have also been proposed to control whitefly infestation, such as using natural predators and parasitoids (e.g. using green lacewing larvae) to control whitefly infestations, or washing of the plants to reduce the number of the pests on the plants. However, also these methods do not provide an optimal solution to the pests, and whitefly remains difficult to control.

Especially in tomato (Solanum lycopersicum) and pepper (Capsicum spp.) whitefly infestations prove to be a problem. Tomato is classified as Solanum sect. Lycopersicon comprising 13 species, of which Solanum lycopersicum is the cultivated tomato, whereas the other 12 species are wild relatives. The genus Capsicum has 25 species of which five are cultivated, including C. annuum, C. chinense, C. baccatum, C. pubescens and C. frutescens. The domestication of tomato and pepper resulted in loss of genetic diversity which makes them prone to abiotic and biotic stresses such as pest attacks. To date, no cultivated tomato and sweet peppers are resistant to whiteflies. Previously several studies have been performed to find whitefly resistance in wild relatives of tomato and sweet pepper. Several wild relatives of tomato (S. pennellii, S. habrochaites, S. peruvianum and S. pimpinellifolium) are known to be more resistant than the cultivated material.

Antibiosis is one of the resistance mechanisms in which a plant exerts an adverse influence on the growth and survival of the insect. One of the most prominent tomato characters that contribute to whitefly antibiosis are trichomes, which are fine outgrowths or appendages on plants, such as glandular hairs. Their function is to secrete metabolites for the plant, including terpenoids, phenylpropanoids, flavonoids, methyl ketones and acyl sugars having diverse functions in the plant related to growth and development, and stress response. For example, mono- and sesquiterpenes, methyl ketones and acyl sugars are secondary metabolites that are known to be associated with whitefly resistance in tomato. Although glandular trichomes seem to play an important role in whitefly resistance, it is actually the compounds within the trichomes that are decisive. A high correlation was found between presence of specific trichomes (type IV trichomes) and whitefly resistance and previous studies showed that whitefly resistance is based on several mechanisms involving many genes and is a complex process. Efforts of introducing whitefly resistance in the cultivated tomato were not successful and new approaches and resistant sources should be considered.

Considering the above, there is a need in the art for tomato plants having an improved insect resistance, more specifically tomato plants having improved insect resistance. In addition, there is a need in the art for a method for providing plants having improved insect resistance, more specifically tomato plants having improved resistance against whiteflies.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a tomato plant having improved whitefly resistance, wherein said plant comprises a combination of an acetyl-CoA-dependent acyltransferase gene (SlAT2) encoding a cDNA sequence having at least 95%, preferably at least 98%, more preferably at least 99% sequence identity with SEQ ID No. 2, and an APETALA2e ethylene-responsive transcription factor gene (AP2e) encoding a cDNA sequence having at least 95%, preferably at least 98%, more preferably at least 99% sequence identity with SEQ ID No. 5, wherein said combination of SlAT2 and AP2e genes result in an increased C₂₉H₄₈O₁₅ and C₃₆H₆₂O₁₅ acyl sugar content as compared to a tomato plant not comprising said combination of genes. Even more preferably the SlAT2 gene encodes a cDNA of SEQ ID No. 2 and the AP2e gene encodes a cDNA of SEQ ID. No. 5.

The tomato plant of present invention, preferably a S. lycopersicum plant has improved insect resistance, wherein the plant comprises a SlAT2 gene in combination with and an AP2e gene. The APETALA2 (AP2) gene family (sometimes also called the AP2/ethylene-responsive element-binding factor (ERF) gene family or ERF/AP2 gene family) defines a large gene family (>100+ genes) of DNA-binding proteins called AP2/ERF in tomato plants. The AP2 genes perform an array of functions including hormone regulation, the establishment of the floral meristem organ identity, and regulation and growth of floral organs and development, as well as various responses to environmental stimuli and stress responses. Furthermore, it is known that various different AP2 genes provide for changes in the ratio of hexose to sucrose during seed development of a plant, and that the AP2 protein regulates the amount of sugars in the system and is involved in transportation, shaping, and signalling in said plant using these various sugars. Surprisingly it was found that the SlAT2 gene in combination with the AP2e gene promotes and regulates the production of the type of specific acyl sugars C₂₉H₄₈O₁₅ (S4:C17 acyl sucrose) and/or C₃₆H₆₂O₁₅ (S4:C24 acyl sucrose), and that these specific increased production of acyl sugars are linked with high levels of insect resistance in the plant. The SlAT2 gene encodes for an acetyl-CoA-dependent acyltransferase enzyme and AP2e genes encode for APETALA2e ethylene-responsive transcription factor that is involved in the regulation of acyl sugars being produced. It seems that AP2e is related to the capacity of producing general amounts of different types of acyl sugars, while the SlAT2 promotes the production of the specific acyl sugars that impact the insect resistance in the plant. The AP2e effects the total amount of acyl sucrose produced by switching on genes involved in the biosynthetic pathway and formation of trichomes, whereas SlAT2 has a strong effect on the final type of acyl sucrose produced. An active SlAT2 enzyme is responsible for adding an additional acetyl-group to acyl sucrose that has already three acyl-groups. This results in an increase of the amount of tetra-acyl sucrose at the expense of tri-acyl sucrose, which results in an improved insect resistance as observed in tomato plants. It is an additional step to a series of reactions that are catalysed by SlAT type of enzymes different from SlAT2; different Acyl-Co transferases which only add up to three acyl chains to the initial sucrose molecule and hence only contribute indirectly to insect resistance.

According to another preferred embodiment, the present invention relates to the tomato plant, wherein said plant comprises tetra-acyl (S4) sugar and tri-acyl (S3) sugars, wherein the ratio between tetra-acyl (S4) sugars and tri-acyl (S3) sugars (S4:S3) in the plant is at least 1, preferably at least 1.2, more preferably at least 1.5, most preferably at least 1.7. Experiments show that the type of acyl sugars is crucial for providing the whitefly resistance in tomato plants. It was observed that plants comprising the AP2e gene and producing acyl sugars are not always resistant for whitefly. However in case the plant also comprise the SlAT2 gene, the plant show improved insect resistance which was shown to be linked to high levels of tetra-acyl (S4) sucroses, for example high C₂₉H₄₈O₁₅ (S4:C17) and C₃₆H₆₂O₁₅ (S4:C24), in comparison to susceptible plants that mostly accumulated tri-acyl sucroses (S3).

The tomato plant of present invention has increased C₂₉H₄₈O₁₅ and/or C₃₆H₆₂O₁₅ acyl sugar content in comparison to a plant that does not comprise the SlAT2 gene in combination with the AP2e gene. The acyl sugars are trichome exudates, more specifically the C₂₉H₄₈O₁₅ (S4:C17 acyl sucrose) and C₃₆H₆₂O₁₅ (S4:C24 acyl sucrose) acyl sugars are type IV trichrome exudates, that cause resistance to insects in tomato plants. AP2e is involved in both trichome development and acyl sugar production. Tomato plants comprising the AP2e gene have an increase of type IV trichomes in the leaf surface and stem. Many trichome exudates of tomato (approx. 90%) are comprised of acyl sugars, of which more than 70 compounds are known. The acyl sugars produced in tomato consist of different combinations of acyl groups, originating from different aliphatic acids with varying chain length and esterified to the hydroxyl groups of glucose or sucrose. The acyl chains are primarily of short to medium chain length aliphatic acids with either a branched or strait chain. In tomato it has been shown that the primary short acyl chains of acyl sugar are either acetate (C2) or branched chain amino acid derived, i.e. 2-methyl-propanoic acid (C4) and 3-methyl-butanoic acid (C5). Longer acyl groups are probably derived from beta-oxidation products of fatty acids. However, the presence and abundance of specific acyl sugars differ significantly between resistant and susceptible plants, wherein specifically C₂₉H₄₈O₁₅ and C₃₆H₆₂O₁₅ acyl sugar content is high in insect resistant plants. These acyl sugars are sticky substances that act as a glue trap and are also toxic for insects, more specifically whiteflies, providing improved insect resistance for the plant. Furthermore, not only whiteflies but other pierce-sucking insects avoid settling on leaves in the presence of these specific acyl sugars.

According to a preferred embodiment, the present invention relates to the tomato plant, wherein the whitefly is one or more selected from the group consisting of Aleurocanthus woglumi (citrus blackfly), Aleyrodes proletella (cabbage whitefly), Bemisia tabaci (silverleaf whitefly), Trialeurodes vaporariorum (greenhouse whitefly), preferably Trialeurodes vaporariorum and/or Bemisia tabaci.

According to a preferred embodiment of the present invention the present plants detailed above are not plants exclusively obtained by means of an essentially biological process.

Although the present genomic regions or fragments can be introduced into tomato plants by introgression, because the nucleotide sequences of the present genomic fragments are known, these genomic fragments, for example, can be artificially constructed in yeast and subsequently allowed to recombine with susceptible tomato genomes. Alternatively, these genomic regions or fragments can be amplified by long-range PCR amplifications and the resulting amplification fragments can be transformed into spinach cells in a single step or in a series of transformations ultimately resulting in the present tomato plants. The present genomic fragments, completely or in parts later to be reassembled, can also be isolated from gels or columns for example after restriction digestion, and subsequently transformed into tomato cells. Furthermore, mutations, deletions or insertions in the genome can be obtained via EMS mutagenesis, and/or CRISPR technology. Yet alternatively, the genomic fragments of interest can be introduced into a vector under a (strong) promotor. Subsequently, susceptible plants can be transformed with the vector and the sequence of interest would be expressed resulting in resistance. These techniques are readily available for the skilled person. Construction of artificial chromosomes comprising the present genomic fragments is also contemplated within the context of the present invention.

According to another preferred embodiment, the present invention relates to the tomato plant, wherein the genomic region encoding the SlAT2 gene having at least 95%, preferably at least 98%, more preferably at least 99% sequence identity with SEQ ID No. 1 and wherein the genomic region encoding the AP2e gene having at least 95%, preferably at least 98%, more preferably at least 99% sequence identity with SEQ ID No. 4. SEQ ID No. 1 and SEQ ID No. 4 are the genomic regions comprising the SlAT2 and AP2e gene respectively including their respective promotor elements. having at least 95%, preferably at least 98%, more preferably at least 99% sequence identity with

According to yet another preferred embodiment, the present invention relates to the tomato plant, wherein the SlAT2 gene encodes for the protein sequence represented by SEQ ID No. 3, and wherein the AP2e gene encodes for the protein sequence represented by SEQ ID No. 6.

According to another preferred embodiment, the present invention relates to the tomato plant, wherein the wherein the SlAT2 gene encodes for the coding sequence of SEQ ID No. 2 and the AP2e gene encodes for the coding sequence of SEQ ID No. 5.

According to yet another preferred embodiment, the present invention relates to the tomato plant, wherein the acyl sugar content of C₂₉H₄₈O₁₅ is at least 150 μg/g fresh weight (FW) of plant leaves, preferably at least 200 μg/g FW of plant leaves, more preferably at least 250 μg/g FW of plant leaves and/or wherein the acyl sugar content of C₃₆H₆₂O₁₅ is at least 125 μg/g FW of plant leaves, preferably at least 175 μg/g FW of plant leaves, more preferably at least 250 μg/g FW of plant leaves. The fresh weight (FW) is the weight of a plant or plant part, in this case plant leaves when harvested. Experiments of the bioassay show that at the claimed acyl sugar concentrations level of resistance is observed of a whitefly mortality of on average 40% or higher.

According to yet another preferred embodiment, the present invention relates to the tomato plant, wherein the plant is obtainable from deposit NCIMB 43748 at NCIMB Ltd, Ferguson Building, Craibstone Estate, Bucksburn, Aberdeen AB21 9YA, Scotland on 18 Mar. 2021.

According to another preferred embodiment, the present invention relates to the tomato plant, wherein the plant furthermore has an increased acyl sugar content of one or more selected from the group consisting of C₂₈H₄₆O₁₅, C₃₄H₅₈O₁₅ and C₃₅H₆₀O₁₅ in comparison to a plant that does not comprise the SlAT2 gene and AP2e gene. Next to the acyl sucrose compounds C₂₉H₄₈O₁₅, C₃₆H₆₂O₁₅, C₂₈H₄₆O₁₅, C₃₄H₅₈O₁₅, C₃₅H₆₀O₁₅, two additional sugars C₃₃H₅₆O₁₅, and C₃₇H₆₄O₁₅ were found to be present in higher concentrations (although less pronounced) in resistant tomato plants in comparison to their susceptible counterparts.

According to yet another preferred embodiment, the present invention relates to the tomato plant, wherein the acyl sugar content of C₂₈H₄₆O₁₅ is at least 10 μg/g fresh weight (FW) of plant leaves, preferably at least 15 μg/g fresh weight (FW) of plant leaves, more preferably at least 20 μg/g fresh weight (FW) of plant leaves, and/or wherein the acyl sugar content of C₃₄H₅₈O₁₅ is at least 15 μg/g fresh weight (FW) of plant leaves, preferably a at least 20 μg/g fresh weight (FW) of plant leaves, more preferably at least 25 μg/g fresh weight (FW) of plant leaves, and/or wherein the acyl sugar content of C₃₅H₆₀O₁₅ is at least 12.5 μg/g fresh weight (FW) of plant leaves, preferably at least 15 μg/g fresh weight (FW) of plant leaves, more preferably at least 20 μg/g fresh weight (FW) of plant leaves.

According to another preferred embodiment, the present invention relates to the tomato plant, wherein the plant is furthermore resistant to mite, preferably spider mite (Tetranychus urticae).

The present invention, according to a second aspect, relates to seeds, fruits or plant part of a tomato plant of present invention.

The present invention, according to a further aspect, relates to a method for providing a tomato plant having improved whitefly resistance, the method comprises the steps of providing an whitefly susceptible tomato plant and mutating its genome comprising;
providing a combination of an acetyl-CoA-dependent acyltransferase gene (SlAT2) encoding a cDNA sequence having at least 95% sequence identity with SEQ ID No. 2, and an APETALA2e ethylene-responsive transcription factor gene (AP2e) encoding a cDNA sequence having at least 95% sequence identity with SEQ ID No. 5, wherein said combination of SlAT2 and AP2e genes result in an increased C₂₉H₄₈O₁₅ and C₃₆H₆₂O₁₅ acyl sugar content as compared to a tomato plant not comprising said combination of genes.

The present invention, according to a further aspect, relates to a method for providing a tomato plant having improved whitefly resistance, wherein the method comprises the steps of;
a) crossing of a tomato plant that is susceptible to whitefly with a whitefly resistant tomato plant of present invention as defined,
b) selecting S. lycopersicum plants having improved insect resistance that comprise the SlAT2 gene and AP2e gene. The selection of tomato plants having improved insect resistance can be based on the determination of C₂₉H₄₈O₁₅ and/or C₃₆H₆₂O₁₅ acyl sugar content, wherein the acyl sugar content of C₂₉H₄₈O₁₅ is at least 150 μg/g fresh weight (FW) of plant leaves, preferably at least 200 μg/g FW of plant leaves, more preferably at least 250 μg/g FW of plant leaves and/or wherein the acyl sugar content of C₃₆H₆₂O₁₅ is at least 125 μg/g FW of plant leaves, preferably at least 175 μg/g FW of plant leaves, more preferably at least 250 μg/g FW of plant leaves. Furthermore, selection of whitefly resistant tomato plants can also be done by determination or identification of the specific sequences (cDNA, gDNA or protein sequences) of SlAT2 and AP2e, as identified herein as SEQ ID NO. 1 to SEQ ID NO. 6.

According to another preferred embodiment, the present invention relates to the method, wherein the selection of S. lycopersicum plants having improved insect resistance is by determination of C₂₉H₄₈O₁₅ and/or C₃₆H₆₂O₁₅ acyl sugar content, wherein the acyl sugar content of C₂₉H₄₈O₁₅ is at least 150 μg/g of fresh weight (FW) of plant leaves and/or wherein the acyl sugar content of C₃₆H₆₂O₁₅ is at least 125 μg/g of fresh weight (FW) of plant leaves.

The present invention, according to a further aspect, relates to a combination of two genomic regions for providing insect resistance in tomato plants, wherein one genomic region comprising SEQ ID No. 1 that encodes an acetyl-CoA-dependent acyltransferase gene (SlAT2) and a second genomic region comprising SEQ ID No. 4 that encodes an APETALA2e ethylene-responsive transcription factor gene (AP2e).

The present invention, according to a further aspect, relates to a combination of two genes for providing insect resistance in tomato plants, wherein one gene encodes an acetyl-CoA-dependent acyltransferase (SlAT2) protein comprising SEQ ID No. 3 and a second gene that encodes an APETALA2e ethylene-responsive transcription factor (AP2e) comprising SEQ ID No. 6.

The present invention, according to a further aspect, relates to the use of a combination of the two genomic regions or two genes as defined above in tomato plants for providing whitefly resistant tomato plants.

The present invention will be further detailed in the following examples and figures wherein:

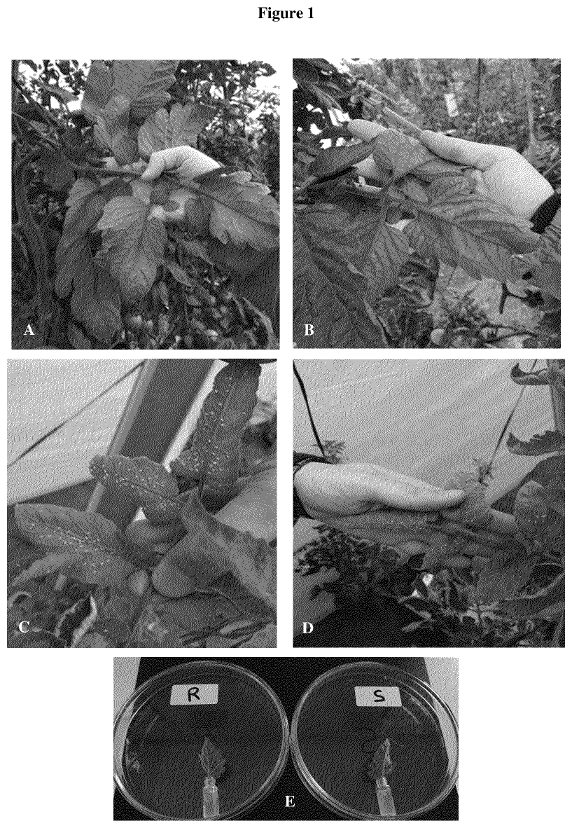

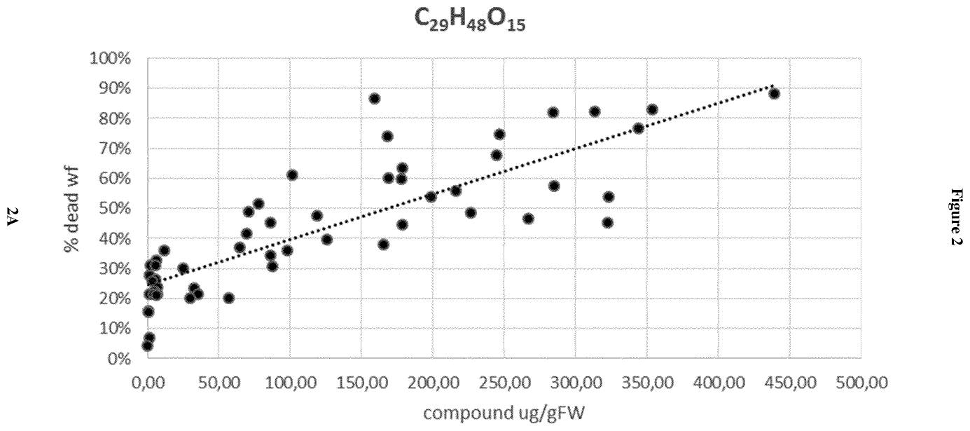

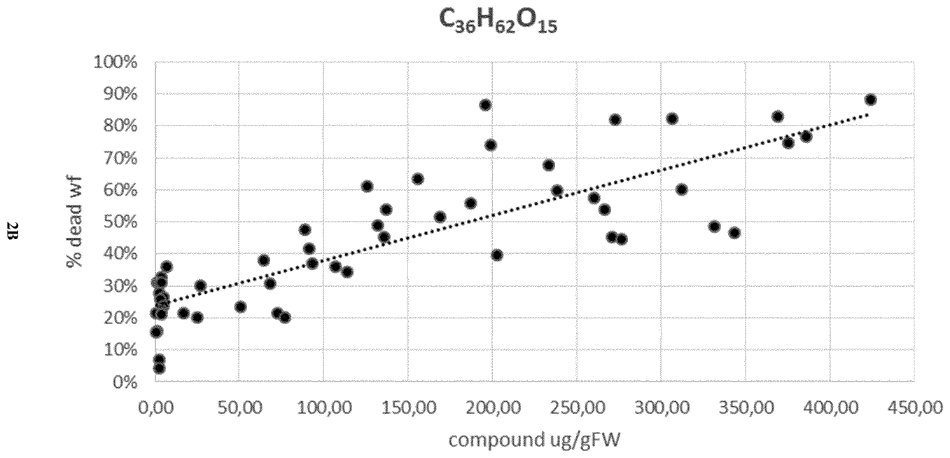

FIG. 1: Shows the leaves of a tomato plant (S. lycopersicum) according to present invention (A and B) and an insect susceptible tomato plant (S. lycopersicum) (C and D). Both tomato plants have been exposed to a whitefly infestation in a commercial greenhouse where a whiteflies infestation was promoted. The leaves of the plants of present invention are free from whitefly infestation, whereas the leaves of the insect susceptible tomato plant are clearly infected by whitefly. Figure E shows the leaves of the insect susceptible tomato plant (S) and of the tomato plant according to present invention (R); it is clear that the whitefly are alive and on the leaf surface of the S plant, whereas on the R plant the whiteflies are dead and absent on the leave surface.

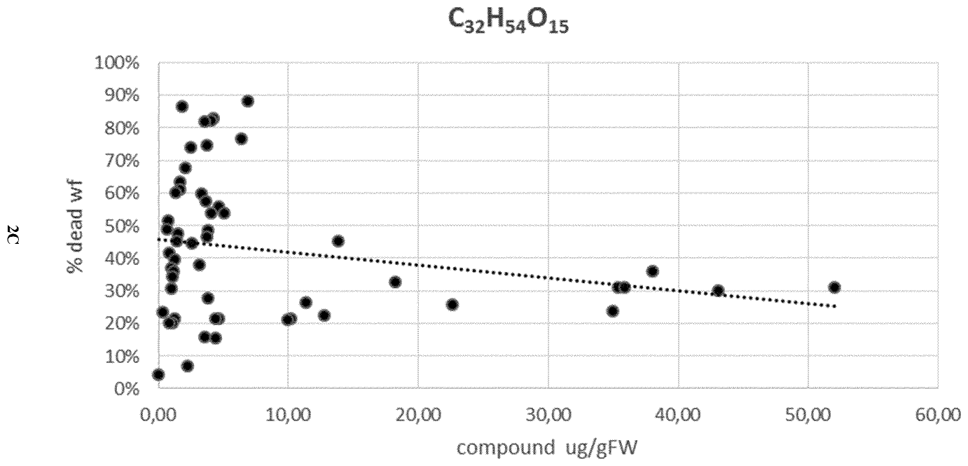

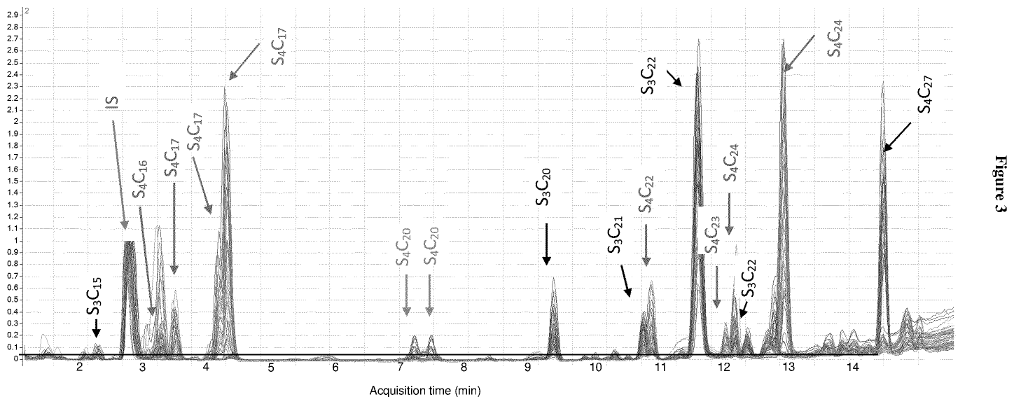

FIG. 2: Shows the ratio of dead whiteflies (WF) in against increasing concentrations of acyl sugar in a tomato plant. The graph 2A shows that there is a linear relationship between the number of dead whiteflies and the concentration of acyl sugar C36H62O15. The graph 2B shows the dose response of C29H48O15 acyl sugar. Both specific acyl sugars show to negatively affect the survival of the whiteflies. On the other hand, other acyl sugars present in the plant like C32H54O15 (graph 2C) does not affect the whiteflies as such. The fresh weight (FW) is the weight of a plant, in this case FW of plant leaves when harvested. The bioassay shows that the level of acyl sugar content in the leaves of the plant directly affects the level of resistance that is observed as whitefly mortality.

FIG. 3: Shows a LC-MS superimposed chromatogram showing the presence and relative concentrations of various acyl sugars of insect resistant plants (red peaks), intermediate resistant plants (orange peaks), and susceptible plants (greenpeaks). The acyl sugar compounds that are labelled in red are present in high concentrations in plants that are insect resistant and are considered to play a vital role in insect resistance in the plant. The acyl sugars labelled in green are mainly present in susceptible plants. From this analysis, it can be concluded that plants having improved insect resistance is linked to high levels of C₂₉H₄₈O₁₅ (S4:C17) and C₃₆H₆₂O₁₅ (S4:C24) acyl sugars. Furthermore, no significant changes were observed in the acyl sugar content of C₂₇H₄₆O₁₄ (S3:C15), C₃₂H₅₆O₁₄ (S3:C20), C₃₃H₅₈O₁₄ (S3:C21), C₃₄H₆₀O₁₄ (S3:C22) and C₃₉H₆₈O₁₅ (S4:C27) in relation to insect resistance of the plants.

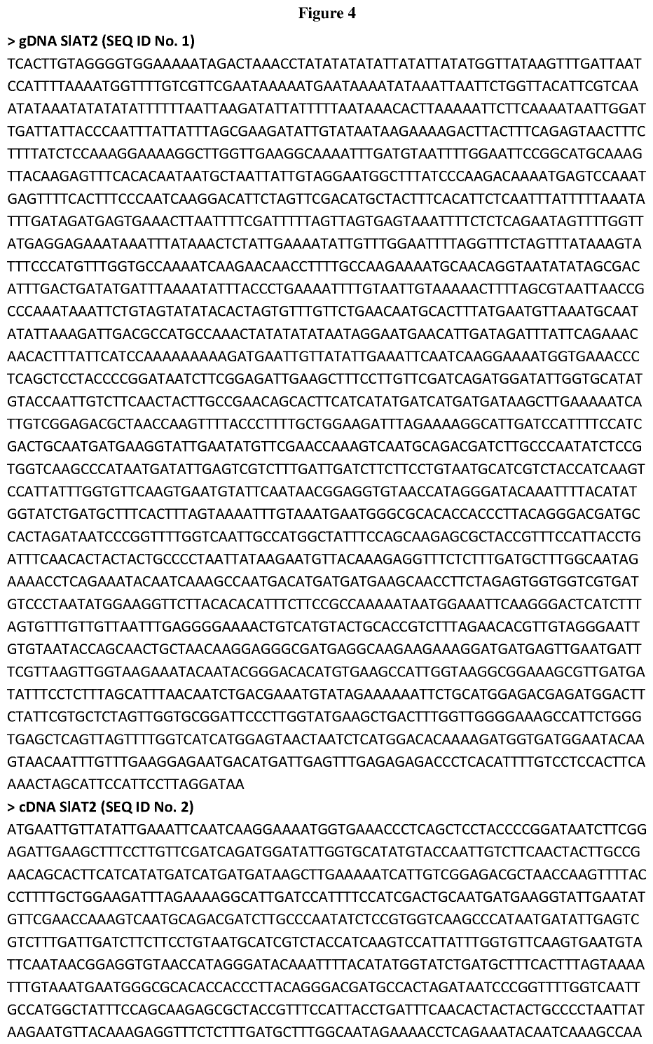

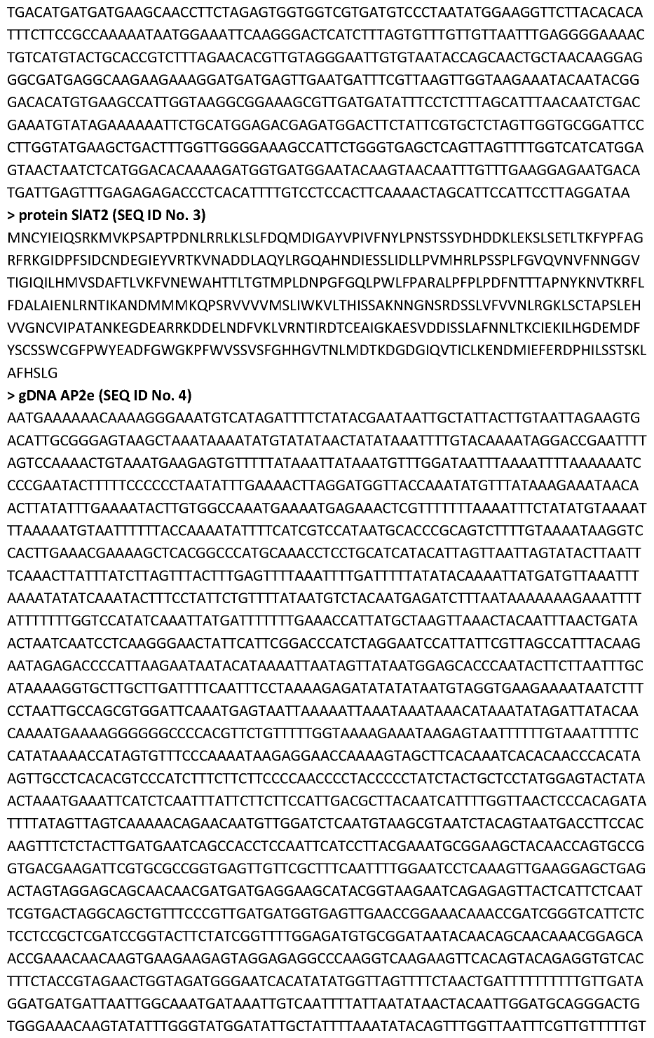

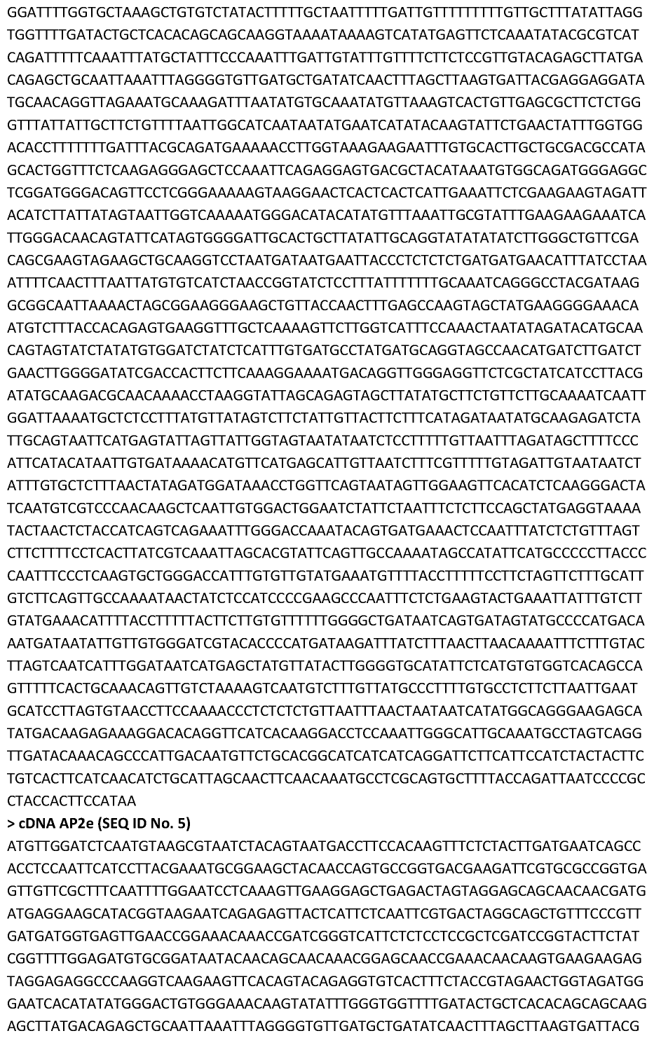

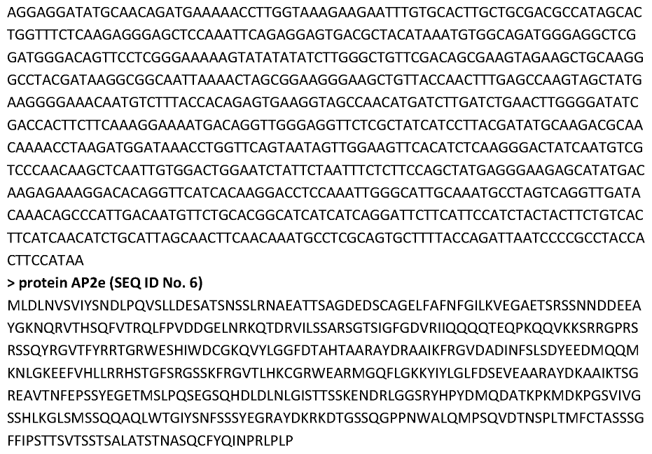

FIG. 4: Shows the genomic region (gDNA), coding sequence (cDNA) and protein sequence of the SlAT2 (SEQ ID No. 1 to 3, respectively), and gDNA, cDNA and protein sequence of the AP2e (SEQ ID No. 4 to 6, respectively), wherein SlAT2 in combination with AP2e provides whitefly resistance in tomato plants.

### EXAMPLES

### Detached Leaves Bioassay

Young leaves of at least 12 weeks old plants (S. lycopersicum) of about 4 cm long were detached from the top of approximately 50 tomato plants grown in a plastic greenhouse. Petiole of the leaf was placed into a tube containing a nutritive agarose gel. The tube, with the adaxial part of the leaf side up, was placed (using blu-tack) in horizontal, on the wall of a glass petri dish at medium height, such that the leaf had space between the leaf and the petri dish on both the adaxial and abaxial sides. Each petri dish was then inoculated with 25 whiteflies, which were anaesthetized with CO₂ for 3 seconds.

After 24-48 hours, the number of whiteflies in the adaxial or abaxial surface was counted together with the number of dead whiteflies. Each plant was tested twice and the number of whiteflies that were alive (feeding from the adaxial and the abaxial part of the leaf plus the whiteflies still flying around in the petri dish) versus the dead whiteflies were used to calculate the percentage of dead whiteflies. Correlation analysis between specific acyl sucroses and the whitefly mortality revealed that different specific acyl sucrose molecules play a different role in the resistance/susceptibility to whiteflies. As showed in FIG. 2 representing whitefly (WF) mortality versus the specific acyl sucrose content present in the plant, mainly C₂₉H₄₈O₁₅ (S4:C17) and C₃₆H₆₂O₁₅ (S4:C24) acyl sugars showed have a high impact on resistance. Other acyl sugar compounds that also showed a link with resistance are the C₂₈H₄₆O₁₅ (S4:C16), C₃₄H₅₈O₁₅ (S4:C22), C₃₅H₆₀O₁₅ (S4:C23), C₃₃H₅₆O₁₅ (S4:C21), and C₃₇H₆₄O₁₅ (S4:C25) acyl sugars.

### Liquid Chromatograpy Mass Spectrometry (LC-MS) Analysis Acyl Sugar Content in Tomato

A chemical analysis by LC-MS of the leaf surface was performed on a set of tomato plants, including insect resistant tomato plants according to present invention, intermediate resistant plants, and susceptible plants (all S. lycopersicum). Plants were grown till 10 side shoots and two opposite leaflets (3×3×3 cm) from the 3^{rd} or 4^{th} apical leaf were put in a 10 ml glass vial. Two milliliters of methanol with internal standard (Sucrose octaacetate, 10 mg/l) was added and the vial was shaken for 15 seconds. The leaflets were taken out and 300 μl of methanol extract was transferred to an LC-vial and analyzed using an Agilent 1290 Infinity II UHPLC coupled to an Agilent 6230 TOF mass spectrometer.

One μL of extract was injected and separated on an Agilent ZORBAX RRHD Eclipse Plus C18 column at 50° C. with a mobile-phase flow-rate of 0.3 m/min. The mobile phase was comprised of water+0.1% formic acid (A) and acetonitrile+0.1% formic acid (B) in the following A:B gradient; from 60:40 to 45:55 in 6 minutes to 10:90 in 8 minutes to 60:40 in 3 minutes. Molecules were ionised at 325 eV (positive mode) and detected in a range of 50-1500 mu at 1 spectrum/second. The extract comprised mainly of acylsugars that were detected as sodium adducts in the mass spectrometer.

Individual acyl sugars were identified using MassHunter Qualitative Analysis software (Agilent) by calculating the molecular formulas on the basis of the mother ion constituting the chromatographic peaks. Here, molecular formulas were constrained by allowing carbon, hydrogen and oxygen atoms to form the mother ion, in combination with H⁺, Na⁺ and K⁺ and formate adducts to appear, plus a double bond equivalent (DBE) range of 1-10. The exact mass of the mother ion in combination with the DBE allows extrapolation of the basic structure of the acyl sugar molecule; the backbone moiety, number of acyl chains and the total number of carbon atoms forming the acyl chains. Amounts of acylsugars were calculated by using MassHunter Quantitative Analysis Software (Agilent) for chromatogram peak integration and comparing the total peak area of the individual acylsugars to that of the internal standard (sucrose octaacetate).

With the LC-MS corresponding results were obtained as obtained with the detached leaves bioassay as described above. Further evidence that specific acyl sugars are involved in the insect resistance can be derived from the LC-MS chromatogram plot, FIG. 3. The individual chromatograms of methanolic leaf dips of insect resistant plants (red), intermediate resistant plants (orange), and susceptible plants (green) were superimposed. The acyl sugar compounds that are labelled in red are present in high concentrations in plants that showed to be highly resistant to whiteflies. In contrast in plants that showed to be susceptible to whitefly, no or only low concentrations of these specific acyl sugars were detected by LC-MS. Furthermore, in respect to the whitefly susceptible plants the acyl sugars that were mainly present are labelled in green, and remarkably were not, or at low concentrations, present in the resistant plants. From this analysis, it can be concluded that plants having improved insect resistance is linked to high C₂₉H₄₈O₁₅ (S4:C17) and C₃₆H₆₂O₁₅ (S4:C24) acyl sugars. No significant changes were observed in the acyl sugar content of C₂₇H₄₆O₁₄ (S3:C15), C₃₂H₅₆O₁₄ (S3:C20), C₃₃H₅₈O₁₄ (S3:C21), C₃₄H₆₀O₁₄ (S3:C22) and C₃₉H₆₈O₁₅ (S4:C27) in insect susceptible and resistant plants.

### Genotypic Analysis, Mapping of SlAT2 and AP2e.

The production of acyl sugars in tomato plants is linked with high levels of insect resistance in the plant. Important is to know which type of acyl sugars are needed for insect resistance. Looking into genotypic data on the resistant tomato plant population (S. lycopersicum) by marker analysis, marker M8 and M5 (Table 1) were used to identify the QTLs that clearly correlate with the production of (C₃₆H₆₂O₁₅) (S4:C24) and (C₂₉H₄₈O₁₅) (S4:C17) acyl sucrose.

Briefly, genomic regions that are linked to the amount of acyl sugars which are produced by type IV trichomes have been mapped on chromosome 6, based on the reference genome SL2.40. It was determined that the region involved in acyl sugar production linked to insect resistance is located between positions 43250794 bp and 43259933 bp. Marker M5 is 100% linked with the amount of acyl sugars (Table 1) being produced. Based on the reference genome SL2.40 and in silico prediction analysis (ITAG 2.3), one gene Solyc06g075510.2 is located in the fine mapped region that encodes for an APETALA2 ethylene-responsive transcription factor (AP2e).

Furthermore, the type of acyl sugars is crucial for providing the whitefly resistance in tomato plants. It was observed that plants comprising the AP2e gene and producing acyl sugars are not always resistant for whitefly. Comparing the acyl sugar profiles of the susceptible and resistant plants, it was concluded that plants having improved insect resistance are linked to high levels of tetra-acyl (S4) sucroses C₂₉H₄₈O₁₅ (S4:C17) and C₃₆H₆₂O₁₅ (S4:C24) in comparison to susceptible plants that mostly accumulate tri-acyl sucroses (S3). Marker M8 is 100% linked with the type of acyl sugars (Table 1), and one specific sequence was mapped on chromosome 1, which encoded a member of the BAHD family of acyltransferases, more specifically an acetyl-CoA-dependent acyltransferase enzyme SlAT2, capable of acyl sucrose acetylation and responsible for the production of C₂₉H₄₈O₁₅ (S4:C17) and C₃₆H₆₂O₁₅ (S4:C24).

Sequencing of the functional SlAT2 gene resulted in a genomic sequence including promotor region (SEQ ID No. 1). Plants comprising SEQ ID No. 1, i.e. a functional SlAT2, in combination with AP2e have an increased S4/S3 ratio and are highly resistant to whitefly compared to the plants without SEQ ID No. 1, these plants are not capable to produce S4 sugars which results in susceptibility to whitefly. SEQ ID No. 1 shows the genomic sequence comprising the SlAT2 gene including promotor region of the whitefly resistant plant of present invention. SEQ ID No. 2 shows the coding sequence of SlAT2 in the plant of present invention that encodes for the SlAT2 protein of SEQ ID No. 3. SEQ ID No. 4 shows the genomic sequence of the AP2e gene including promotor region of the whitefly resistant plant of present invention. SEQ ID No. 5 shows the coding sequence of AP2e in the plant of present invention that encodes for the AP2e protein of SEQ ID No. 6.

TABLE 1
Marker sequences used for QTL mapping

| Marker | Sequence |
|---|---|
| M5_F (SEQ ID No. 7) | GCGAGGCATTTGTTGAAGTTGC |
| | TAATGC |
| M5_R (SEQ ID No. 8) | GGTTGATACAAACAGCCCATTG |
| M8_F (SEQ ID No. 9) | AAGCAATGCGAAATATCGTAAC |
| M8_R (SEQ ID No. 10) | GAGAGACCCTCACATTTTGTC |

It was found that the SlAT2 in combination with the AP2e gene specifically promotes and regulates the production of specific types of acyl sugars like C₂₉H₄₈O₁₅ (S4:C17 acyl sucrose) and/or C₃₆H₆₂O₁₅ (S4:C24 acyl sucrose) and more in general increases the ration between tetra- (S4) and tri-acylated (S3) sugar. The combination AP2e (marker M5)+SlAT2 (marker M8) increases the level of S4:C17 and S4:C24 acyl sucrose that is needed for whitefly resistance. Several tomato plants were selected for their presence of AP2e gene and the presence/absence of SlAT2 gene, homo/heterozygous using the M5 and M8 markers. Total acyl sugars content (g per gram plant fresh weight (gFW) per plant was determined, as well as the presence of specifically specific acyl sugars C₂₉H₄₈O₁₅ (S4:C17 acyl sucrose) and C₃₆H₆₂O₁₅ (S4:C24 acyl sucrose), the ratio between tetra- (S4) and tri-acylated (S3) sucroses, and the resistance to whitefly. The genotypes of SlAT2 are co-segregating with the production of S4:C17 and S4:C24 acyl sucrose and increased S4/S3 ratio, and is linked with the white fly resistance level, see Table 2.

TABLE 2
AP2e, SIAT2 presence in plants and the effect on
Acyl sugars and whitefly resistance.

| | | | Total | Sum S4C17 + | Ratio | WF |
|---|---|---|---|---|---|---|
| | AP2e | SIAT2 | AcS | S4C24 | S4:S3 | resistance |
| Plant | (M5) | (M8) | (μg/gFW) | (μg/gFW) | AcS | % |
| 1. | b | b | 1038.7 | 479.7 | 1.46 | 60 |
| 2. | b | b | 1503.8 | 551.9 | 2.33 | 49 |
| 3. | b | b | 1060.5 | 431.5 | 1.67 | 44 |
| 4. | b | a | 823.1 | 9.6 | 0.05 | 21 |
| 5. | b | a | 579.8 | 5.6 | 0.33 | 7 |
| 6. | b | a | 1872.4 | 12.6 | 0.33 | 22 |
| 7. | b | b | 1735.8 | 544.3 | 1.95 | 74 |
| 8. | b | h | 1316.5 | 350.4 | 1.12 | 60 |
| 9. | b | h | 1820.4 | 632.2 | 1.87 | 45 |
| 10. | b | a | 2090.5 | 59.4 | 0.48 | 30 |
| 11. | b | a | 1180.0 | 28.7 | 0.57 | 21 |

a = absent
b = present
h = heterozygous

1. A tomato plant having improved whitefly resistance, wherein said plant comprises a combination of an acetyl-CoA-dependent acyltransferase gene (SlAT2) encoding a cDNA sequence having at least 95% sequence identity with SEQ ID No. 2, and an APETALA2e ethylene-responsive transcription factor gene (AP2e) encoding a cDNA sequence having at least 95% sequence identity with SEQ ID No. 5, wherein said combination of SlAT2 and AP2e genes result in an increased C₂₉H₄₈O₁₅ and C₃₆H₆₂O₁₅ acyl sugar content as compared to a tomato plant not comprising said combination of genes.

2. The tomato plant according to claim 1, wherein said tomato plant comprises tetra-acyl (S4) sugar and tri-acyl (S3) sugars, wherein a ratio between tetra-acyl (S4) sugars and tri-acyl (S3) sugars (S4:S3) in the tomato plant is at least 1.

3. The tomato plant according to claim 1, wherein the genomic region encoding the SlAT2 gene comprises SEQ ID No. 1 and wherein the genomic region encoding the AP2e gene comprises SEQ ID No. 4.

4. The tomato plant according to claim 1, wherein the SlAT2 gene encodes for the protein sequence represented by SEQ ID No. 3, and wherein the AP2e gene encodes for the protein sequence represented by SEQ ID No. 6.

5. The tomato plant according to claim 1, wherein the acyl sugar content of C₂₉H₄₈O₁₅ is at least 150 μg/g of fresh weight (FW) of plant leaves, and/or wherein the acyl sugar content of C₃₆H₆₂O₁₅ is at least 125 μg/g of FW of plant leaves.

6. The tomato plant according to claim 1, wherein the plant furthermore has an increased acyl sugar content of one or more selected from the group consisting of C₂₈H₄₆O₁₅, C₃₄H₅₈O₁₅ and C₃₅H₆₀O₁₅ as compared to a tomato plant not comprising said combination of genes.

7. The tomato plant according to claim 1, wherein the acyl sugar content of C₂₈H₄₆O₁₅ is at least 10 μg/g of FW of plant leaves and/or wherein the acyl sugar content of C₃₄H₅₈O₁₅ is at least 15 μg/g of FW of plant leaves, and/or wherein the acyl sugar content of C₃₅H₆₀O₁₅ is at least 12.5 μg/g of FW of plant leaves.

8. The tomato plant according to claim 1, wherein the plant is obtainable from deposit NCIMB 43748.

9. The tomato plant according to claim 1, wherein the plant is furthermore resistant to mites.

10. A seed, a fruit, or a plant part of the tomato plant according to claim 1.

11. A method for providing a tomato plant having improved whitefly resistance comprising the steps of:
providing a whitefly susceptible tomato plant; and
mutating its genome by introducing therein a combination of an acetyl-CoA-dependent acyltransferase gene (SlAT2) encoding a cDNA sequence-having at least 95% sequence identity with SEQ ID No. 2, and an APETALA2e ethylene-responsive transcription factor gene (AP2e) encoding a cDNA sequence having at least 95% sequence identity with SEQ ID No. 5,
wherein said combination of SlAT2 and AP2e genes result in an increased C₂₉H₄₈O₁₅ and C₃₆H₆₂O₁₅ acyl sugar content as compared to a tomato plant not comprising said combination of genes.

12. A method for providing a tomato plant having improved whitefly resistance, wherein the method comprises the steps of:
a) crossing a tomato plant that is susceptible to whitefly with the tomato plant according to claim 1; and
b) selecting S. lycopersicum plants having improved insect resistance that comprise the SlAT2 gene and AP2e gene.

13. The method according to claim 12, wherein the selection of S. lycopersicum plants having improved insect resistance is by determination of C₂₉H₄₈O₁₅ and/or C₃₆H₆₂O₁₅ acyl sugar content, wherein the acyl sugar content of C₂₉H₄₈O₁₅ is at least 150 μg/g of fresh weight (FW) of plant leaves and/or wherein the acyl sugar content of C₃₆H₆₂O₁₅ is at least 125 μg/g of fresh weight (FW) of plant leaves.

14. A combination of two genomic regions for providing insect resistance in tomato plants, wherein one genomic region comprising SEQ ID No. 1 that encodes an acetyl-CoA-dependent acyltransferase gene (SlAT2) and a second genomic region comprising SEQ ID No. 4 that encodes an APETALA2e ethylene-responsive transcription factor gene (AP2e).

15. A combination of two genes for providing insect resistance in tomato plants, wherein one gene encodes an acetyl-CoA-dependent acyltransferase (SlAT2) protein comprising SEQ ID No. 3 and a second gene that encodes an APETALA2e ethylene-responsive transcription factor (AP2e) comprising SEQ ID No. 6.

16. (canceled)

17. The tomato plant according to claim 1, wherein a ratio between tetra-acyl (S4) sugars and tri-acyl (S3) sugars (S4:S3) in the tomato plant is at least 1.7.

18. The tomato plant according to claim 1, wherein the tomato plant is furthermore resistant to spider mites.


SEQUENCE LISTING (10 sequences)

SEQ ID NO: 1 - DNA, 2322 bp, Solanum lycopersicum
  gDNA SlAt2
tcacttgtaggggtggaaaaatagactaaacctatatatatattatattatatggttataagtttgattaatccattttaaaatggttttgtcgttcgaataaaaatgaataaaatataaattaattctggttacattcgtcaaatataaatatatatattttttaattaagatattatttttaataaacacttaaaaattcttcaaaataattggattgattattacccaatttattatttagcgaagatattgtataataagaaaagacttactttcagagtaactttcttttatctccaaaggaaaaggcttggttgaaggcaaaatttgatgtaattttggaattccggcatgcaaagttacaagagtttcacacaataatgctaattattgtaggaatggctttatcccaagacaaaatgagtccaaatgagttttcactttcccaatcaaggacattctagttcgacatgctactttcacattctcaatttatttttaaatatttgatagatgagtgaaacttaattttcgatttttagttagtgagtaaattttctctcagaatagttttggttatgaggagaaataaatttataaactctattgaaaatattgtttggaattttaggtttctagtttataaagtatttcccatgtttggtgccaaaatcaagaacaaccttttgccaagaaaatgcaacaggtaatatatagcgacatttgactgatatgatttaaaatatttaccctgaaaattttgtaattgtaaaaacttttagcgtaattaaccgcccaaataaattctgtagtatatacactagtgtttgttctgaacaatgcactttatgaatgttaaatgcaatatattaaagattgacgccatgccaaactatatatataataggaatgaacattgatagatttattcagaaacaacactttattcatccaaaaaaaaagatgaattgttatattgaaattcaatcaaggaaaatggtgaaaccctcagctcctaccccggataatcttcggagattgaagctttccttgttcgatcagatggatattggtgcatatgtaccaattgtcttcaactacttgccgaacagcacttcatcatatgatcatgatgataagcttgaaaaatcattgtcggagacgctaaccaagttttacccttttgctggaagatttagaaaaggcattgatccattttccatcgactgcaatgatgaaggtattgaatatgttcgaaccaaagtcaatgcagacgatcttgcccaatatctccgtggtcaagcccataatgatattgagtcgtctttgattgatcttcttcctgtaatgcatcgtctaccatcaagtccattatttggtgttcaagtgaatgtattcaataacggaggtgtaaccatagggatacaaattttacatatggtatctgatgctttcactttagtaaaatttgtaaatgaatgggcgcacaccacccttacagggacgatgccactagataatcccggttttggtcaattgccatggctatttccagcaagagcgctaccgtttccattacctgatttcaacactactactgcccctaattataagaatgttacaaagaggtttctctttgatgctttggcaatagaaaacctcagaaatacaatcaaagccaatgacatgatgatgaagcaaccttctagagtggtggtcgtgatgtccctaatatggaaggttcttacacacatttcttccgccaaaaataatggaaattcaagggactcatctttagtgtttgttgttaatttgaggggaaaactgtcatgtactgcaccgtctttagaacacgttgtagggaattgtgtaataccagcaactgctaacaaggagggcgatgaggcaagaagaaaggatgatgagttgaatgatttcgttaagttggtaagaaatacaatacgggacacatgtgaagccattggtaaggcggaaagcgttgatgatatttcctctttagcatttaacaatctgacgaaatgtatagaaaaaattctgcatggagacgagatggacttctattcgtgctctagttggtgcggattcccttggtatgaagctgactttggttggggaaagccattctgggtgagctcagttagttttggtcatcatggagtaactaatctcatggacacaaaagatggtgatggaatacaagtaacaatttgtttgaaggagaatgacatgattgagtttgagagagaccctcacattttgtcctccacttcaaaactagcattccattccttaggataa

SEQ ID NO: 2 - DNA, 1356 bp, Solanum lycopersicum
  cDNA SlAt2
atgaattgttatattgaaattcaatcaaggaaaatggtgaaaccctcagctcctaccccggataatcttcggagattgaagctttccttgttcgatcagatggatattggtgcatatgtaccaattgtcttcaactacttgccgaacagcacttcatcatatgatcatgatgataagcttgaaaaatcattgtcggagacgctaaccaagttttacccttttgctggaagatttagaaaaggcattgatccattttccatcgactgcaatgatgaaggtattgaatatgttcgaaccaaagtcaatgcagacgatcttgcccaatatctccgtggtcaagcccataatgatattgagtcgtctttgattgatcttcttcctgtaatgcatcgtctaccatcaagtccattatttggtgttcaagtgaatgtattcaataacggaggtgtaaccatagggatacaaattttacatatggtatctgatgctttcactttagtaaaatttgtaaatgaatgggcgcacaccacccttacagggacgatgccactagataatcccggttttggtcaattgccatggctatttccagcaagagcgctaccgtttccattacctgatttcaacactactactgcccctaattataagaatgttacaaagaggtttctctttgatgctttggcaatagaaaacctcagaaatacaatcaaagccaatgacatgatgatgaagcaaccttctagagtggtggtcgtgatgtccctaatatggaaggttcttacacacatttcttccgccaaaaataatggaaattcaagggactcatctttagtgtttgttgttaatttgaggggaaaactgtcatgtactgcaccgtctttagaacacgttgtagggaattgtgtaataccagcaactgctaacaaggagggcgatgaggcaagaagaaaggatgatgagttgaatgatttcgttaagttggtaagaaatacaatacgggacacatgtgaagccattggtaaggcggaaagcgttgatgatatttcctctttagcatttaacaatctgacgaaatgtatagaaaaaattctgcatggagacgagatggacttctattcgtgctctagttggtgcggattcccttggtatgaagctgactttggttggggaaagccattctgggtgagctcagttagttttggtcatcatggagtaactaatctcatggacacaaaagatggtgatggaatacaagtaacaatttgtttgaaggagaatgacatgattgagtttgagagagaccctcacattttgtcctccacttcaaaactagcattccattccttaggataa

SEQ ID NO: 3 - protein, 451 aa, Solanum lycopersicum
  Protein SlAT2
MNCYIEIQSRKMVKPSAPTPDNLRRLKLSLFDQMDIGAYVPIVFNYLPNSTSSYDHDDKLEKSLSETLTKFYPFAGRFRKGIDPFSIDCNDEGIEYVRTKVNADDLAQYLRGQAHNDIESSLIDLLPVMHRLPSSPLFGVQVNVFNNGGVTIGIQILHMVSDAFTLVKFVNEWAHTTLTGTMPLDNPGFGQLPWLFPARALPFPLPDFNTTTAPNYKNVTKRFLFDALAIENLRNTIKANDMMMKQPSRVVVVMSLIWKVLTHISSAKNNGNSRDSSLVFVVNLRGKLSCTAPSLEHVVGNCVIPATANKEGDEARRKDDELNDFVKLVRNTIRDTCEAIGKAESVDDISSLAFNNLTKCIEKILHGDEMDFYSCSSWCGFPWYEADFGWGKPFWVSSVSFGHHGVTNLMDTKDGDGIQVTICLKENDMIEFERDPHILSSTSKLAFHSLG

SEQ ID NO: 4 - DNA, 4556 bp, Solanum lycopersicum
  gDNA AP2e
aatgaaaaaacaaaagggaaatgtcatagattttctatacgaataattgctattacttgtaattagaagtgacattgcgggagtaagctaaataaaatatgtatataactatataaattttgtacaaaataggaccgaattttagtccaaaactgtaaatgaagagtgtttttataaattataaatgtttggataatttaaaattttaaaaaatccccgaatactttttcccccctaatatttgaaaacttaggatggttaccaaatatgtttataaagaaataacaacttatatttgaaaatacttgtggccaaatgaaaatgagaaactcgtttttttaaaatttctatatgtaaaatttaaaaatgtaattttttaccaaaatattttcatcgtccataatgcacccgcagtcttttgtaaaataaggtccacttgaaacgaaaagctcacggcccatgcaaacctcctgcatcatacattagttaattagtatacttaatttcaaacttatttatcttagtttactttgagttttaaattttgatttttatatacaaaattatgatgttaaatttaaaatatatcaaatactttcctattctgttttataatgtctacaatgagatctttaataaaaaaagaaattttatttttttggtccatatcaaattatgatttttttgaaaccattatgctaagttaaactacaatttaactgataactaatcaatcctcaagggaactattcattcggacccatctaggaatccattattcgttagccatttacaagaatagagaccccattaagaataatacataaaattaatagttataatggagcacccaatacttcttaatttgcataaaaggtgcttgcttgattttcaatttcctaaaagagatatatataatgtaggtgaagaaaataatctttcctaattgccagcgtggattcaaatgagtaattaaaaattaaataaataaacataaatatagattatacaacaaaatgaaaaggggggccccacgttctgtttttggtaaaagaaataagagtaattttttgtaaatttttccatataaaaccatagtgtttcccaaaataagaggaaccaaaagtagcttcacaaatcacacaacccacataagttgcctcacacgtcccatctttcttcttccccaacccctaccccctatctactgctcctatggagtactataactaaatgaaattcatctcaatttattcttcttccattgacgcttacaatcattttggttaactcccacagatattttatagttagtcaaaaacagaacaatgttggatctcaatgtaagcgtaatctacagtaatgaccttccacaagtttctctacttgatgaatcagccacctccaattcatccttacgaaatgcggaagctacaaccagtgccggtgacgaagattcgtgcgccggtgagttgttcgctttcaattttggaatcctcaaagttgaaggagctgagactagtaggagcagcaacaacgatgatgaggaagcatacggtaagaatcagagagttactcattctcaattcgtgactaggcagctgtttcccgttgatgatggtgagttgaaccggaaacaaaccgatcgggtcattctctcctccgctcgatccggtacttctatcggttttggagatgtgcggataatacaacagcaacaaacggagcaaccgaaacaacaagtgaagaagagtaggagaggcccaaggtcaagaagttcacagtacagaggtgtcactttctaccgtagaactggtagatgggaatcacatatatggttagttttctaactgattttttttttgttgataggatgatgattaattggcaaatgataaattgtcaattttattaatataactacaattggatgcagggactgtgggaaacaagtatatttgggtatggatattgctattttaaatatacagtttggttaatttcgttgtttttgtggattttggtgctaaagctgtgtctatactttttgctaatttttgattgtttttttttgttgctttatattaggtggttttgatactgctcacacagcagcaaggtaaaataaaagtcatatgagttctcaaatatacgcgtcatcagatttttcaaatttatgctatttcccaaatttgattgtatttgttttcttctccgttgtacagagcttatgacagagctgcaattaaatttaggggtgttgatgctgatatcaactttagcttaagtgattacgaggaggatatgcaacaggttagaaatgcaaagatttaatatgtgcaaatatgttaaagtcactgttgagcgcttctctgggtttattattgcttctgttttaattggcatcaataatatgaatcatatacaagtattctgaactatttggtggacacctttttttgatttacgcagatgaaaaaccttggtaaagaagaatttgtgcacttgctgcgacgccatagcactggtttctcaagagggagctccaaattcagaggagtgacgctacataaatgtggcagatgggaggctcggatgggacagttcctcgggaaaaagtaaggaactcactcactcattgaaattctcgaagaagtagattacatcttattatagtaattggtcaaaaatgggacatacatatgtttaaattgcgtatttgaagaagaaatcattgggacaacagtattcatagtggggattgcactgcttatattgcaggtatatatatcttgggctgttcgacagcgaagtagaagctgcaaggtcctaatgataatgaattaccctctctctgatgatgaacatttatcctaaattttcaactttaattatgtgtcatctaaccggtatctcctttatttttttgcaaatcagggcctacgataaggcggcaattaaaactagcggaagggaagctgttaccaactttgagccaagtagctatgaaggggaaacaatgtctttaccacagagtgaaggtttgctcaaaagttcttggtcatttccaaactaatatagatacatgcaacagtagtatctatatgtggatctatctcatttgtgatgcctatgatgcaggtagccaacatgatcttgatctgaacttggggatatcgaccacttcttcaaaggaaaatgacaggttgggaggttctcgctatcatccttacgatatgcaagacgcaacaaaacctaaggtattagcagagtagcttatatgcttctgttcttgcaaaatcaattggattaaaatgctctcctttatgttatagtcttctattgttacttctttcatagataatatgcaagagatctattgcagtaattcatgagtattagttattggtagtaatataatctcctttttgttaatttagatagcttttcccattcatacataattgtgataaaacatgttcatgagcattgttaatctttcgtttttgtagattgtaataatctatttgtgctctttaactatagatggataaacctggttcagtaatagttggaagttcacatctcaagggactatcaatgtcgtcccaacaagctcaattgtggactggaatctattctaatttctcttccagctatgaggtaaaatactaactctaccatcagtcagaaatttgggaccaaatacagtgatgaaactccaatttatctctgtttagtcttcttttcctcacttatcgtcaaattagcacgtattcagttgccaaaatagccatattcatgcccccttaccccaatttccctcaagtgctgggaccatttgtgttgtatgaaatgttttacctttttccttctagttctttgcattgtcttcagttgccaaaataactatctccatccccgaagcccaatttctctgaagtactgaaattatttgtcttgtatgaaacattttacctttttacttcttgtgttttttggggctgataatcagtgatagtatgccccatgacaaatgataatattgttgtgggatcgtacaccccatgataagatttatctttaacttaacaaaatttctttgtacttagtcaatcatttggataatcatgagctatgttatacttggggtgcatattctcatgtgtggtcacagccagtttttcactgcaaacagttgtctaaaagtcaatgtctttgttatgcccttttgtgcctcttcttaattgaatgcatccttagtgtaaccttccaaaaccctctctctgttaatttaactaataatcatatggcagggaagagcatatgacaagagaaaggacacaggttcatcacaaggacctccaaattgggcattgcaaatgcctagtcaggttgatacaaacagcccattgacaatgttctgcacggcatcatcatcaggattcttcattccatctactacttctgtcacttcatcaacatctgcattagcaacttcaacaaatgcctcgcagtgcttttaccagattaatccccgcctaccacttccataa

SEQ ID NO: 5 - DNA, 1425 bp, Solanum lycopersicum
  cDNA AP2e
atgttggatctcaatgtaagcgtaatctacagtaatgaccttccacaagtttctctacttgatgaatcagccacctccaattcatccttacgaaatgcggaagctacaaccagtgccggtgacgaagattcgtgcgccggtgagttgttcgctttcaattttggaatcctcaaagttgaaggagctgagactagtaggagcagcaacaacgatgatgaggaagcatacggtaagaatcagagagttactcattctcaattcgtgactaggcagctgtttcccgttgatgatggtgagttgaaccggaaacaaaccgatcgggtcattctctcctccgctcgatccggtacttctatcggttttggagatgtgcggataatacaacagcaacaaacggagcaaccgaaacaacaagtgaagaagagtaggagaggcccaaggtcaagaagttcacagtacagaggtgtcactttctaccgtagaactggtagatgggaatcacatatatgggactgtgggaaacaagtatatttgggtggttttgatactgctcacacagcagcaagagcttatgacagagctgcaattaaatttaggggtgttgatgctgatatcaactttagcttaagtgattacgaggaggatatgcaacagatgaaaaaccttggtaaagaagaatttgtgcacttgctgcgacgccatagcactggtttctcaagagggagctccaaattcagaggagtgacgctacataaatgtggcagatgggaggctcggatgggacagttcctcgggaaaaagtatatatatcttgggctgttcgacagcgaagtagaagctgcaagggcctacgataaggcggcaattaaaactagcggaagggaagctgttaccaactttgagccaagtagctatgaaggggaaacaatgtctttaccacagagtgaaggtagccaacatgatcttgatctgaacttggggatatcgaccacttcttcaaaggaaaatgacaggttgggaggttctcgctatcatccttacgatatgcaagacgcaacaaaacctaagatggataaacctggttcagtaatagttggaagttcacatctcaagggactatcaatgtcgtcccaacaagctcaattgtggactggaatctattctaatttctcttccagctatgagggaagagcatatgacaagagaaaggacacaggttcatcacaaggacctccaaattgggcattgcaaatgcctagtcaggttgatacaaacagcccattgacaatgttctgcacggcatcatcatcaggattcttcattccatctactacttctgtcacttcatcaacatctgcattagcaacttcaacaaatgcctcgcagtgcttttaccagattaatccccgcctaccacttccataa

SEQ ID NO: 6 - protein, 474 aa, Solanum lycopersicum
  Protein AP2e
MLDLNVSVIYSNDLPQVSLLDESATSNSSLRNAEATTSAGDEDSCAGELFAFNFGILKVEGAETSRSSNNDDEEAYGKNQRVTHSQFVTRQLFPVDDGELNRKQTDRVILSSARSGTSIGFGDVRIIQQQQTEQPKQQVKKSRRGPRSRSSQYRGVTFYRRTGRWESHIWDCGKQVYLGGFDTAHTAARAYDRAAIKFRGVDADINFSLSDYEEDMQQMKNLGKEEFVHLLRRHSTGFSRGSSKFRGVTLHKCGRWEARMGQFLGKKYIYLGLFDSEVEAARAYDKAAIKTSGREAVTNFEPSSYEGETMSLPQSEGSQHDLDLNLGISTTSSKENDRLGGSRYHPYDMQDATKPKMDKPGSVIVGSSHLKGLSMSSQQAQLWTGIYSNFSSSYEGRAYDKRKDTGSSQGPPNWALQMPSQVDTNSPLTMFCTASSSGFFIPSTTSVTSSTSALATSTNASQCFYQINPRLPLP

SEQ ID NO: 7 - DNA, 28 bp, Artificial Sequence
  M5 forward
gcgaggcatttgttgaagttgctaatgc

SEQ ID NO: 8 - DNA, 22 bp, Artificial Sequence
  M5 reverse
ggttgatacaaacagcccattg

SEQ ID NO: 9 - DNA, 22 bp, Artificial Sequence
  M8 forward
aagcaatgcgaaatatcgtaac

SEQ ID NO: 10 - DNA, 21 bp, Artificial Sequence
  M8 reverse
gagagaccctcacattttgtc